(19) United States
(12) Patent Application Publication
(10) Pub. No.: US 2002/0031765 A1
(43) Pub. Date: Mar. 14, 2002

(54) OLIGONUCLEOTIDES FOR IDENTIFYING PRECURSORS OF AMIDATED POLYPEPTIDE HORMONES

(76) Inventors: JEAN MARTINEZ, SAUSSAN (FR); CATHERINE GOZE, MONTPELLIER (FR)

Correspondence Address: CHARLES A MUSERLIAN, BIERMAN MUSERLIAN AND LUCAS, 600 THIRD AVENUE, NEW YORK, NY, 10016, US

(21) Appl. No.: 09/486,142

(22) PCT Filed: Aug. 7, 1998
(86) PCT No.: PCT/FR98/01767

(30) Foreign Application Priority Data
Aug. 26, 1997 (FR) 97/10643

(51) Int. Cl. C12P 19/34; C12Q 1/68; C07H 21/02; C07H 21/04; C07K 1/00; C07K 14/00; C07K 17/00

(52) U.S. Cl. 435/6; 435/91.2; 536/23.1; 530/350

(57) ABSTRACT

The invention concerns novel oligonucleotides and their application as probes for identifying RNAm's coding for precursors of amidated polypeptide hormones and, thereby, identifying novel amidated polypeptide hormones. The invention also concerns oligonucleotides of the disclosed nucleotide sequence and a method for identifying precursors of amidated hormones.

The present invention relates to new oligonucleotides and their use as probes for identification of the mRNA which codes for precursors of amidated polypeptide hormones, and to the identification of new amidated polypeptide hormones. The invention thus relates to oligonucleotides of which the nucleotide sequence is described below and a method for identification of precursors of hormones.

Amidated polypeptide hormones are synthesized in the form of a precursor which undergoes maturation. This maturation consists of an amidation reaction.

The amidation reaction of the C-terminal end is a characteristic reaction of amidated polypeptide hormones. This reaction, which occurs on the precursor of one or more hormones, allows maturation of the hormone and also ensures its biostability in the physiological medium: the amide group formed is less vulnerable than the free acid function. The hormone is therefore more resistant to carboxypeptidases, it remains active in the cell for longer and retains an optimum affinity for its receptor site.

Amidation has been widely described (“Peptide amidation”, Alan F. Bradbury and Derek G. Smyth, TIBS 16:112-115, March 1991 and “Functional and structural characterization of peptidylamidoglycolate lyase, the enzyme catalysing the second step in peptide amidation”, A. G. Katopodis, D. S. Ping, C. E. Smith and S. W. May, Biochemistry, 30(25):6189-6194, June 1991), and its mechanism is as follows:

1—Cleavage of the precursor polypeptide chain of the hormone by an endoprotease at the two basic amino acids, that is to say arginine and/or lysine,

2—Subsequently two cleavages by carboxypeptidase result, which lead to the extended glycine intermediate,

3—The enzyme PAM (peptidyl-glycine-α-amidating monooxygenase) comprises two distinct enzymatic activities: firstly, it converts the extended glycine intermediate into an α-hydroxyglycine derivative, the subunit of the enzyme PAM involved is PHM (peptidyl-glycine-α-hydroxylating monooxygenase). The derivative obtained serves as the substrate for the second subunit of PAM (called PAL: peptidyl-α-hydroxyglycine-α-amidating lyase), which fixes the amine function of the glycine on to the amino acid immediately adjacent to the N-terminal side and liberates glyoxylate.

This reaction involves the presence of a recognition site on the precursor of the hormone or hormones, a site which always comprises the sequence: glycine and two basic amino acids (arginine or lysine) (cf. A. G. Katopodis et coll., Biochemistry, 30(25), 6189-6194, June 1991, and references cited).

The amidated polypeptide hormones which are to be secreted outside the endoplasmic reticulum are known to comprise a consensus signal sequence of about fifteen to thirty amino acids, this sequence being present at the N-terminal end of the polypeptide chain. It is cut later by a signal peptidase enzyme such that it is no longer found in the protein once secreted (cf F. Cuttitta, The Anatomical Record, 236, 87-93 (1993) and references cited).

At the present time, the discovery of a new protein is not easy. Proteins can be isolated and purified by various techniques: precipitation at the isoelectric point, selective extraction by certain solvents and then purification by crystallization, counter-current distribution, adsorption, partition or ion exchange chromatography, electrophoresis . . . . However, these techniques imply knowledge of the properties of the protein to be isolated. Furthermore, if a pure sample of a new protein of interest at the therapeutic level is available, there are still several stages before a genetically modified microorganism capable of synthesizing it is available.

The method proposed by the present invention offers the advantage, by using a characteristic of the peptide sequence of the precursor of all amidated hormones known to date, of allowing simultaneous detection of several new hormones of this category. This search is affected by direct identification of the nucleotide sequence which codes for the said precursors in cDNA banks prepared from tissues in which the precursors of these hormones can be synthesized.

The search by this method is much less restricting than the abovementioned conventional techniques of biochemistry, since:

it can lead to the isolation of several distinct precursors present in the same tissue by the same principle;

it allows detection, under the same technical conditions, of precursors corresponding to hormones which have very different biochemical and biological properties;

it allows concomitant identification of all the peptide hormones which can be contained in the same precursor.

As a result, this invention allows a not insignificant saving in time and money in a sector where the costs of research and development represent a very high proportion of turnover.

The present invention will also allow pharmacological study of active substances having a fundamental physiological roll in the mammalian organism: hormones and more particularly amidated polypeptide neurohormones. Having available for the first time cDNA corresponding to active substances, it will then be possible to introduce the cloned vector by genetic engineering to lead to synthesis of hormones having a therapeutic use by means of microorganisms.

The invention first relates to a single-stranded oligonucleotide OX which can hybridize under mild conditions with an oligonucleotide OY of the sequence Y1-Y2-Y3-Y4-Y5, in which Y1 represents a nucleotide sequence of 1 to 12 nucleotides or Y1 is suppressed, Y2 represents a trinucleotide which codes for Gly, Y3 and Y4 independently represent a trinucleotide which codes for Arg or Lys and Y5 represents a nucleotide sequence of 1 to 21 nucleotides or Y5 is suppressed.

Nucleotide is understood as meaning a monomeric unit of RNA or DNA having the chemical structure of a nucleoside phosphoric ester. A nucleoside results from bonding of a purine base (purine, adenine, guanine or analogues) or of a pyrimidine base (pyrimidine, cytosine, uracil or analogues) with ribose or deoxyribose. An oligonucleotide is a polymer of nucleotides designating a primer sequence, a probe or a fragment of RNA or DNA.

The oligonucleotides mentioned can be obtained by synthesis, and there is a reference automated method which is described in the following publications: “DNA synthesis” by S. A. Narang, Tetrahedron, 39, 3 (1983) and “Synthesis and use of synthetic oligonucleotides” by K. Itakura, J. J. Rossi and R. B. Wallace, Annu. Rev. Biochem., 53, 323 (1984).

Preferably, OX can hybridize with OY under stringent conditions.

More preferably, OX can hybridize with an oligonucleotide OY of the sequence Y2-Y3-Y4-Y5.

Still more preferably, OX can hybridize with an oligonucleotide OY of the sequence Y1-Y2-Y3-Y4 or Y2-Y3-Y4.

In particular, OX can hybridize with an oligonucleotide OY such that Y5 represents a nucleotide sequence Y6-Y7-Y8-Y9, in which Y6 represents a trinucleotide which codes for Ser, Thr or Tyr, Y7 represents a trinucleotide which codes for any amino acid, Y8 represents a trinucleotide which codes for Glu or Asp and Y9 represents a nucleotide sequence comprising 1 to 12 nucleotides. More particularly, OX can hybridize with an oligonucleotide OY such that Y1 and Y9 are suppressed.

Especially particularly, OX can hybridize with an oligonucleotide OY in which Y2 represents a trinucleotide which codes for Gly, Y3 represents a trinucleotide which codes for Lys, Y4 represents a trinucleotide which codes for Arg and Y5 represents a sequence of 3 trinucleotides which codes for Ser-Ala-Glu.

This sequence was determined with the aid of a statistical study of 27 known amidation sites and led to definition of a given pattern of amino acids over 6 positions: Gly-Lys-Arg-Ser-Ala-Glu.

Because of the degeneration of the genetic code and the high number of codons corresponding to Gly (4 codons), Arg (6 codons) and Ser (6 codons), the oligonucleotide sequence was constructed with the aid of two procedures which allow this degeneration to be taken into account:

use of certain positions of inosine, a nucleotide in which the nitrogen base hypoxanthine pairs indiscriminately with the 4 nitrogen bases which make up the DNA,

variation at certain positions of the nature of the nitrogen base incorporated, thus generating a number of combinations of oligonucleotides proportional to the number of different bases introduced.

The present invention also relates to an oligonucleotide OY comprising 9 to 42 nucleotides of the sequence Y1-Y2-Y3-Y4-Y5, in which Y1 represents a nucleotide sequence of 1 to 12 nucleotides or Y1 is suppressed, Y2 represents a trinucleotide which codes for Gly, Y3 and Y4 independently represent a trinucleotide which codes for Arg or Lys and Y5 represents a nucleotide sequence of 1 to 21 nucleotides or Y5 is suppressed.

Preferably, the invention relates to an oligonucleotide OY such that Y1 is suppressed or such that Y5 is suppressed.

The invention particularly relates to an oligonucleotide OY such that Y5 represents a nucleotide sequence Y6-Y7-Y8-Y9, in which Y6 represents a trinucleotide which codes for Ser, Thr or Tyr, Y7 represents a trinucleotide which codes for any amino acid, Y8 represents a trinucleotide which codes for Glu or Asp and Y9 represents a nucleotide sequence comprising 1 to 12 nucleotides.

The invention more particularly relates to an oligonucleotide OY such that Y1 and Y9 are suppressed.

The invention especially particularly relates to an oligonucleotide OY, characterized in that Y1 is suppressed, Y2 represents a trinucleotide which codes for Gly, Y3 represents a trinucleotide which codes for Lys, Y4 represents a trinucleotide which codes for Arg and Y5 represents a sequence of three trinucleotides which codes for Ser-Ala-Glu.

The present invention also relates to a single-stranded oligonucleotide OZ, characterized in that it comprises 15 to 39 nucleotides and is capable of hybridizing with a consensus signal sequence characteristic of amidated polypeptide hormones, the said sequence having as the formula Z1-Z2-Z3-Z4-Z5-Z6-Z7, in which Z1 represents a nucleotide sequence of 1 to 12 nucleotides or Z1 is suppressed, Z2 and Z3 represent two trinucleotides which code for Leu, Z4 and Z5 represent two trinucleotides which code for any two amino acids, Z6 represents a trinucleotide which codes for Leu and Z7 represents a nucleotide sequence of 1 to 12 nucleotides or Z7 is suppressed.

In this invention, hormone will be understood as meaning amidated polypeptide hormones of the endocrine system, and more particularly neurohormones.

The consensus signal sequence is a sequence carried by the precursors of proteins which are secreted by cells after their maturation.

Finally, the present invention relates to a group of oligonucleotides OX or OZ such as constitutes a combinatorial library.

In the invention described, combinatorial library is understood as meaning a group of oligonucleotides synthesized by taking as the model a nucleotide sequence which codes for a sequence of amino acids of which some can be varied. Because of the degeneration of the genetic code, a group of different oligonucleotides will be obtained.

The invention also relates to a method for identification of the precursor of a peptide having an amidated C-terminal end, characterized by the following successive stages:

1—Obtaining of a DNA bank;

2—Hybridization of one or more oligonucleotides OX with the said DNA bank;

3—Identification of the DNA sequence or sequences of the said bank which hybridizes with an oligonucleotide OX;

4—Identification in this sequence or sequences of one or more peptides with a possible amidated C-terminal end.

A method such that the DNA bank is a cDNA bank will be preferred.

Complementary DNA (cDNA) is a nucleotide chain of which the sequence is complementary to that of an mRNA, the reaction leading to monocatenated cDNA being catalysed by inverse transcriptase. Bicatenated cDNA can be obtained by the action of DNA polymerase, and is then inserted with the aid of a ligase into a plasmid or a vector derived from λ bacteriophage.

A cDNA bank contains the cDNA corresponding to the cytoplasmic mRNA extracted from a given cell. The bank is called complete if it comprises at least one bacterial clone for each starting mRNA.

Hybridization takes place if two oligonucleotides have substantially complementary nucleotide sequences, and they can combine over their length by establishing hydrogen bonds between complementary bases.

A method such that the oligonucleotide OX can be detected with the aid of a marking agent, such as ³²P or digoxigenin, will be particularly preferred.

The agents for radioactive marking of nucleotides most usually used are the elements which emit β-rays, for example ³H, ¹²C, ³²P, ³³P and ³⁵S.

Marking of the oligonucleotide is effected by addition of a phosphate group carried by (γ-³²P)-ATP on to its 5′ end, this reaction being catalysed by the enzyme T4-polynucleotide kinase. Marking by digoxigenin is immunoenzymatic, the digoxigenin being combined with a nitrogen base and incorporated into the oligonucleotide. Its presence is revealed by using an antibody directed against digoxigenin and coupled to an alkaline phosphatase. The presence is revealed using the colour developed by a substrate hydrolysed by the alkaline phosphatase.

Other marking techniques can be employed: oligonucleotides modified chemically so that they contain a metal-complexing agent (complexes of lanthanide are often used), a group containing biotin or acridine ester, a fluorescent compound (fluorescein, rhodamine, Texas red) or others.

A method for identification of the precursor of the aniidated polypeptide hormone such that the hybridization stage uses a combinatorial library of oligonucleotides OX will be especially particularly preferred.

The invention also relates to a method for identification of the precursor of a peptide having an amidated C-terminal end, which comprises the following stages:

1—Obtaining of a DNA bank;

2—Use of the PCR technique to amplify the fragment of interest with the aid of a group of oligonucleotides OX and another group of oligonucleotides OZ;

3—Identification of the DNA sequence of the said bank which hybridizes with the oligonucleotide OX and which has been amplified by the PCR reaction;

4—Identification in this sequence of one or more peptides with a possible amidated C-terminal end.

Fragment of interest is understood as meaning the cDNA sequence which codes for the precursor of one or more amidated polypeptide hormones.

The reaction of amplification of the DNA by a PCR (polymerase chain reaction) requires a DNA preparation denatured by heating at 95° C. This preparation is then paired with an excess of two complementary oligonucleotides at opposite strands of the DNA, on both sides of the sequence to be amplified. Each oligonucleotide then serves as a primer for a DNA polymerase (extracted from thermophilic bacteria of the type Thermus aquatitus: Taq polymerase) for copying each of the strands of the DNA. This cycle can be repeated in an automated manner by successive denaturations-renaturations.

There are numerous references detailing PCR protocols: U.S. Pat. Nos. 4,683,192, 4,683,202, 4,800,159 and 4,965,188, “PCR technology : principles and applications for DNA amplification”, H. Erlich, ed. Stockton Press, New York (1989) and “PCR protocols: a guide to methods and applications”, Innis et al., eds. Academic Press, San Diego, Calif. (1990).

Preferably, the said DNA bank is a cDNA bank.

More preferably, the said oligonucleotide OX can be detected with aid of a marking agent, such as ³²P or digoxigenin.

A method for identification of the precursor of an amidated polypeptide hormone such that the amplification stage uses a combinatorial library of oligonucleotides OX and another combinatorial library of oligonucleotides OZ will be particularly preferred.

The invention also relates to a method for identification of the precursor of a peptide having an amidated C-terminal end, which comprises the following stages:

1—Obtaining of a DNA bank;

2—Use of the PCR technique to amplify the fragment of interest with the aid of a group of oligonucleotides OX;

3—Identification of the DNA sequence of the said bank which hybridizes with the oligonucleotide OX and which has been amplified by the PCR reaction;

4—Identification in this sequence of one or more peptides with a possible amidated C-terminal end.

The aim of this method is to characterize the nucleotide sequences which code for precursors having more than one amidation site.

Preferably, the said DNA bank is a cDNA bank.

More preferably, the said oligonucleotide OX can be detected with the aid of a marking agent, such as ³²P or digoxigenin.

A method for the identification of the precursor of an amidated polypeptide hormone such that the amplification stage uses a combinatorial library of oligonucleotides OX will be particularly preferred.

Another method proposed by the present invention for identification of the precursor of a polypeptide having an amidated C-terminal end is characterized by the following stages:

1—Obtaining of a DNA bank;

2—Use of the PCR technique to amplify the fragment of interest with the aid of an oligonucleotide OX and another single-stranded oligonucleotide capable of hybridizing under mild or stringent conditions with a universal consensus sequence contained in the sequence of the plasmid vector in which the DNA of the said DNA bank are cloned, such as the primers T3, T7, KS, SK, M13, Reverse;

3—Identification of the DNA sequence of the said bank which hybridizes with an oligonucleotide OX;

4—Identification in this sequence of one or more peptides with a possible amidated C-terminal end.

The universal consensus sequence is a sequence carried by the vector in which the DNA of the bank is cloned. This sequence can serve as a primer for the sequencing. The nucleotide sequences of these primers are available in: Sambrook, J., Fritsch, E. F., Maniatis, T., “Molecular cloning, a laboratory manual”, 2nd edition, 1989, Colel Spring Harbor Laboratory Press.

The PCR reaction requires that two oligonucleotides are fixed on to the cDNA cloned in a vector for its amplification to have taken place. In the case where only a single sequence belonging to the DNA fragment to be amplified is known, a solution to overcome this problem is to use an oligonucleotide which could hybridize with a nucleotide sequence belonging to the vector in which the cDNA has been cloned, such as a universal consensus sequence.

Preferably, the said DNA bank is a cDNA bank.

An oligonucleotide OY which can be detected with aid of a marking agent, such as ³²P or digoxigenin, will be preferred.

An amplification stage using a combinatonal library of oligonucleotides OX will be more particularly preferred.

### EXAMPLE

The method described by the invention has been validated by its application to a hormone which has already been isolated. The neurohormone chosen is cholecystokinin (CCK), which is the neuromediator which quantitatively is represented the most in the brain.

1.1. Preparation of the DNA matrix used for PCR reactions from a commercial bank Lambda Zapp II (Rat Brain cDNA Library Vector ref. 936 501) of STRATAGENE (Lafolla. USA).

This Stratagene cDNA bank contains the cloning of the cDNA of the cells of the rat brain.

1.1.1. Release of the cloned cDNA in the form of Bluescript phagemids (Stratagene, Lajolla, U.S.A.).

This is carried out in accordance with the following protocol: 250 μl of the cDNA bank at 2.10⁸ PFU/ml, 200 μl of XL₁ blue bacteria (genotype: recA1 endA1 gyrA96 thi-1 hsdR17 supE44 relA1 lac [F′ proAB lacI^{q}ZΔM15 Tn10 (Tet^{r})]^{c}—cf. Bullock, Fernandez, Short, Biotechniques, 5, 376-379 (1987)—optical density at 600 nm: OD=2.5) and 1 μl of the phage ExAssist™ (cf. Hay, B., Short, J., Strategies, 5, 16-18 (1992)) at 10¹⁰ PFU/ml are brought into contact for 15 minutes at 37° C. The entire system is then incubated on 50 ml of LB medium (composition: 10 g NaCl, 5 g yeast extract and 10 g Bactotryptone per 1 liter of sterile physiological water are mixed) for 3 hours while stirring at 37° C. The culture broth is centrifuged and the supernatant is then activated by heating at 70° C. for 20 minutes.

1.1.2. Obtaining of the cDNA in the form of a double-stranded plasmid bank.

This stage requires 15 minutes of incubation at 37° C. of 100 μl of the inactivated supernatant and 200 μl of SOLR™ bacteria (genotype:e14⁻(McrA⁻) Δ(mcrCB-hsdSMR-mrr)171 sbcC recB recJ uvrC umuC::Tn5 (Kan^{r}) lac gyrA96 relA1 thi-1 endA1 λ^{R} [F′ proAB lacI^{q}ZΔM15]^{c} Su⁻ (nonsuppressing)—cf. Hay, B., Short, J. M., Strategies, 5(1), 16-18 (1992)—OD=1 to 600 nm). After addition of 50 μl ampicillin (at 100 mg/ml) and 50 ml of LB medium, the entire system is incubated at 37° C. while stirring for one night. The plasmids are prepared from 50 ml of culture with the QIAGEN Plasmid Midi Kit protocol and columns from QIAGEN (the QIAGEN columns contain an anion exchange resin with positively charged diethylaminoethanol groups on its surface which interact with the phosphates of the DNA skeleton). A DNA solution at 1.37 μg/μl was thus obtained.

1.2. Amplification of a portion of the precursor of CCK from the plasmid bank thus prepared.

1.2.1. Establishing the sequences of the two oligonucleotides necessary for the PCR reaction.

One of these two nucleotides will contain the sequence complementary to that which codes for the amidation site of CCK, which site is known and has as the sequence Gly-Arg-Arg-Ser-Ala-Glu. This oligonucleotide, which will be called oligo CCK amide, has as its nucleotide sequence:

5′CTCAGCACTGCGCCGGCC 3′

The second oligonucleotide, called oligo CCK 5′, corresponds to the consensus signal sequence:

5′GTGTGTCTGTGCGTGGTG 3′

The size of the expected amplification product is 315 base pairs, which is the distance between the sequences corresponding to these two oligonucleotides on the precursor sequence of the CCK.

1.2.2. PCR reaction.

A dilution D1 containing 1 μl of the enzyme Taq polymerase Goldstar 5 U/μl (cf. Reynier, P., Pellissier, J. F., Harle, J. R., Malthiéry, Y., Biochemical and Biophysical Research Communications, 205(1), 375-380 (1994)), 1 μl of a buffer concentrated 10-fold in standard Taq polymerase and 8 μl water is prepared.

1 μl oligo CCK 5′ at 250 ng/μl, 1 μl oligo CCK amide at 250 ng/μl, 1 μl dNTP at 10 mM each, 1 μl of the cDNA bank at 250 ng/μl, 5 μl of buffer concentrated 10-fold in the enzyme Taq polymerase, 2 μl MgCl₂ at 25 mM, 1 μl of the dilution D1 and 37 μl water are then mixed.

The amplification conditions are the following: heat treatment is first carried out for 5 minutes at 95° C., and 30 cycles are then repeated. The denaturations are carried out at 95° C. for 45 seconds, the hybridization at 60° C. for 30 seconds and the elongation at 72° C. for 1 minute. Finally, a supplementary cycle is conducted with an elongation at 72° C. for 10 minutes.

1.2.3. Results.

The results are read by migration on agarose gel at 0.8% of 1/10 of the product of the PCR reaction. In the presence of 3,8-diamino-5-ethyl-6-phenylphenanthridinium bromide (ethidium bromide), a single intense band of a size slightly greater than the marker of molecular weight 300 is visualized.

1.3. Subcloning of the PCR product into a vector which allows sequencing

The vector used is pGEM T-easy Vector (marketed by PROGEMA Corporation, Madison, USA, ref A 1380—sequence given in appendix I). The stages are the following:

purification of the band corresponding to the PCR product by electroelution,

ligation for one night 16° C. with 1 μl of the vector pGEM T-easy at 50 ng/μl and 1 μl of ligase buffer concentrated 10-fold,

3 μl of product extracted from the purified band, estimated at 20 ng/μl,

topped up to 10 μl with water.

JM 109 bacteria (genotype: e14-(McrA-) recA1 endA1 gyrA96 thi-1 hsdR17(r_{K}-m_{K+}) supE44 relA1 Δ(lac-proAB) [F′ traD36 proAB lacI^{q}ZΔM15]—cf. Yanish-Perron, C., Viera, J., Messing, J., Gene, 33, 103-199 (1985)) are rendered competent by a treatment beforehand with CaCl₂ and are then transformed by a thermal shock of 45 seconds at 42° C. with ⅕ of the ligation. The cells are then cultured on LB-ampicillin medium in a Petri dish overnight at 37° C.

The plasmid DNA of some recombinant clones are prepared. The subcloning is then verified by enzymatic digestion with Eco RI.

1.4 Sequencing

This is carried out by the conventional technique of dideoxynucleotides of SANGER on the vector pGEM T-easy Vector, the PCR product of 315 base pairs having been incorporated (prepared on a large scale using the QIAGEN tip 100 kit). The primer used for the sequencing is the universal oligonucleotide T7 present on the pGEM T-easy Vector plasmid.

1.5. Result.

The following crude sequence is obtained:

GTG TGT CTG TGC GTG GTG ATG GCA GTC CTG GCA GCA
GGC GCC CTG
GCG CAG CCG GTA GTC CCT GTA GAA GCT GTG GAC CCT
ATG GAG CAG
CGG GCG GAG GAG GCG CCC CGA AGG CAG CTG AGG GCT
GTG CTC CGA
CCG GAC AGC GAG CCC CGA GCG CGC CTG GGC GCA CTG
CTA GCC CGA
TAC ATC CAG CAG GTC CGC AAA GCT CCC TCT GGC CGC
ATG TCC GTT
CTT AAG AAC CTG CAG GGC CTG GAC CCT AGC CAC AGG
ATA AGT GAC
CGG GAC TAC ATG GGC TGG ATG GAT TTC GGC CGG CGC
AGT GCT GAG

Translation of the sequence obtained into amino acids results in:

| | | | |
|---|---|---|---|
| VCLCVV | MAVLAAGALA | QPVVPVEAVD | PMEQRAEEAP |
| RRQLRAVLRP | DSEPRARLGA | LLARYIQQVR | KAPSGRMSVL |
| KNLQGLDPSH | RISDRDYMGW | MDFGRRSAE | |

which enables the nucleotide sequence of the precursor of CCK (the sequence of which has been provided by the Swiss databank prot no. p01355) to be easily found.

The amino acids have the following abbreviations:

| | | | |
|---|---|---|---|
| Alanine | A | Leucine | L |
| Argine | R | Lysine | K |
| Aspartic acid | D | Methionine | M |
| Asparagine | N | Phenylalanine | F |
| Cysteine | C | Proline | P |
| Glutamic acid | E | Serine | S |
| Glutamine | Q | Threonine | T |
| Glycine | G | Tryptophan | W |
| Histidine | H | Tyrosine | Y |
| Isoleucine | I | Valine | V |

APPENDIX 1

Sequence of the pGEM ®-T Easy Vector plasmid
The pGEM ®-T Easy Vector plasmid, the sequence of which
is reproduced below, was linearized with EcoR V at base 60 of
this sequence (indicated by an asterisk). A T with
two 3′ends was added to it. The T added is not included in
this sequence. The sequence reproduced below corresponds to
the RNA synthesized by T7 RNA polymerase and is complementary
to the RNA synthesized with SP6 RNA polymerase.

| | |
|---|---|
| 1 | GGGCGAATTG GGCCCGACGT CGCATGCTCC CGGCCGCCAT GGCGGCCGCG |
| 51 | GGAATTCGAT*ATCACTAGTG AATTCGCGGC CGCCTGCAGG TCGACCATAT |
| 101 | GGGAGAGCTC CCAACGCGTT GGATGCATAG CTTGAGTATT CTATAGTGTC |
| 151 | ACCTAAATAG CTTGGCGTAA TCATGGTCAT AGCTGTTTCC TGTGTGAAAT |
| 201 | TGTTATCCGC TCACAATTCC ACACAACATA CGAGCCGGAA GCATAAAGTG |
| 251 | TAAAGCCTGG GGTGCCTAAT GAGTGAGCTA ACTCACATTA ATTGCGTTGC |
| 301 | GCTCACTGCC CQCTTTCCAG TCGGGAAACC TGTCGTGCCA GCTGCATTAA |
| 351 | TGAATCGGCC AACGCGCGGG GAGAGGCGGT TTGCGTATTG GGCGCTCTTC |
| 401 | CGCTTCCTCG CTCACTGACT CGCTGCGCTC GGTCGTTCGG CTGCGGCGAG |
| 451 | CGGTATCAGC TCACTCAAAG GCGGTAATAC GGTTATCCAC AGAATCAGGG |
| 501 | GATAACGCAG GAAAGAACAT GTGAGCAAAA GGCCAGCAAA AGGCCAGGAA |
| 551 | CCGTAAAAAG GCCGCGTTGC TGGCGTTTTT CCATAGGCTC CGCCCCCCTG |
| 601 | ACGAGCATCA CAAAAATCGA CGCTCAAGTC AGAGGTGGCG AAACCCGACA |
| 651 | GGACTATAAA GATACCAGGC GTTTCCCCCT GGAAGCTCCC TCGTGCGCTC |
| 701 | TCCTGTTCCG ACCCTGCCGC TTACCGGATA CCTGTCCGCC TTTCTCCCTT |
| 751 | CGGGAAGCGT GGCGCTTTCT CATAGCTCAC GCTGTAGGTA TCTCAGTTCG |
| 801 | GTGTAGGTCG TTCGCTCCAA GCTGGGCTGT GTGCACGAAC CCCCCGTTCA |
| 851 | GCCCGACCGC TGCGCCTTAT CCGGTAACTA TCGTCTTGAG TCCAACCCGG |
| 901 | TAAGACACGA CTTATCGCCA CTGGCAGCAG CCACTGGTAA CAGGATTAGC |
| 951 | AGAGCGAGGT ATGTAGGCGG TGCTACAGAG TTCTTGAAGT GGTGGCCTAA |
| 1001 | CTACGGCTAC ACTAGAAGGA CAGTATTTGG TATCTGCGCT CTGCTGAAGC |
| 1051 | CAGTTACCTT CGGAAAAAGA GTTGGTAGCT CTTGATCCGG CAAACAAACC |
| 1101 | ACCGCTGGTA GCGGTGGTTT TTTTGTTTGC AAGCAGCAGA TTACGCGCAG |
| 1151 | AAAAAAAGGA TCTCAAGAAG ATCCTTTGAT CTTTTCTACG GGQTCTGACG |
| 1201 | CTCAGTGGAA CGAAAACTCA CGTTAAGGGA TTTTGGTCAT GAGATTATCA |
| 1251 | AAAAGGATCT TCACCTAGAT CCTTTTAAAT TAAAAATGAA GTTTTAAATC |
| 1301 | AATCTAAAGT ATATATGAGT AAACTTGGTC TGACAGTTAC CAATGCTTAA |
| 1351 | TCAGTGAGGC ACCTATCTCA GCGATCTGTC TATTTCGTTC ATCCATAGTT |
| 1401 | GCCTGACTCC CCGTCGTGTA GATAACTACG ATACGGGAGG GCTTACCATC |
| 1451 | TGGCCCCAGT GCTGCAATGA TACCOCQAGA CCCACGCTCA CCGGCTCCAG |
| 1501 | ATTTATCAGC AATAAACCAG CCAGCCGGAA GGGCCQAGCG CAGAAGTGGT |
| 1551 | CCTGCAACTT TATCCGCCTC CATCCAGTCT ATTAATTGTT GCCGGQAAGC |
| 1601 | TAGAGTAAGT AGTTCGCCAG TTAATAGTTT GCGCAACGTT GTTGGCATTG |
| 1651 | CTACAGGCAT CGTGGTGTCA CGCTCGTCGT TTGGTATGGC TTCATTCAGC |
| 1701 | TCCGGTTCCC AACGATCAAG GCGAGTTACA TGATCCCCCA TGTTGTGCAA |
| 1751 | AAAAGCGGTT AGCTCCTTCG GTCCTCCGAT CGTTGTCAGA AGTAAGTTGG |
| 1801 | CCGCAGTGTT ATCACTCATG GTTATGGCAG CACTGCATAA TTCTCTTACT |
| 1851 | GTCATGCCAT CCGTAAGATG CTTTTCTGTG ACTGGTGAGT ACTCAACCAA |
| 1901 | GTCATTCTGA GAATAGTGTA TGCGGCGACC GAGTTGCTCT TGCCCGGCGT |
| 1951 | CAATACGGGA TAATACCGCG CCACATAGCA GAACTTTAAA AGTGCTCATC |
| 2001 | ATTGGAAAAC GTTCTTCGGG GCGAAAACTC TCAAGGATCT TACCGCTGTT |
| 2051 | GAGATCCAGT TCGATGTAAC CCACTCGTGC ACCCAACTGA TCTTCAGCAT |
| 2101 | CTTTTACTTT CACCAGCGTT TCTGGGTGAG CAAAAACAGG AAGGCAAAAT |
| 2151 | GCCGCAAAAA AGGGAATAAG GGCGACACGG AAATGTTGAA TACTCATACT |
| 2201 | CTTCCTTTTT CAATATTATT GAAGCATTTA TCAGGGTTAT TGTCTCATGA |
| 2251 | GCGGATACAT ATTTGAATGT ATTTAGAAAA ATAAACAAAT AGGGGTTCCG |
| 2301 | CGCACATTTC CCCGAAAAGT GCCACCTGTA TGCGGTGTGA AATACCGCAC |
| 2351 | AGATGCGTAA GGAGkAAATA CCGCATCAGG CGAAATTGTA AACGTTAATA |
| 2401 | TTTTGTTAAA ATTCGCGTTA AATATTTGTT AAATCAGCTC ATTTTTTAAC |
| 2451 | CAATAGGCCG AAATCGGCAA AATCCCTTAT AAATCAAAAG AATAGACCGA |
| 2501 | GATAGGGTTG AGTGTTGTTC CAGTTTGGAA CAAGAGTCCA CTATTAAAGA |
| 2551 | ACGTGGACTC CAACGTCAAA GGGCGAAAAA CCGTCTATCA GGGCGATGGC |
| 2601 | CCACTACGTG AACCATCACC CAAATCAAGT TTTTTGCGGT CGAGGTGCCG |
| 2651 | TAAAGCTCTA AATCGGAACC CTAAAGGGAG CCCCCGATTT AGAGCTTGAC |
| 2701 | GGGGAAAGCC GGCGAACGTG GCGAGAAAGG AAGGGAAGAA AGCGAAAGGA |
| 2751 | GCGGGCGCTA GGGCGCTGGC AAGTGTAGCG GTCACGCTGC GCGTAACCAC |
| 2801 | CACACCCGCC GCGCTTAATG CGCCGCTACA GQGCGCGTCC ATTCGCCATT |
| 2851 | CAGGCTGCGC AACTGTTGGG AAGGGCGATC GGTGCGGGCC TCTTCGCTAT |
| 2901 | TACGCCAGCT GGCGAAAGGG GGATGTGCTG CAAGGCGATT AAGTTGGGTA |
| 2951 | ACGCCAGGGT TTTCCCAGTC ACGACGTTGT AAAACGACGG CCAGTGAATT |
| 3001 | GTAATACGAC TCACTATA |

1. Single-stranded oligonucleotide OX, characterized in that it comprises 9 to 42 nucleotides and is capable of hybridizing under mild conditions with an oligonucleotide OY of the sequence Y1-Y2-Y3-Y4-Y5, in which Y1 represents a nucleotide sequence of 1 to 12 nucleotides or Y1 is suppressed, Y2 represents a trinucleotide which codes for Gly, Y3 and Y4 independently represent a trinucleotide which codes for Arg or Lys and Y5 represents a nucleotide sequence of 1 to 21 nucleotides or Y5 is suppressed.

2. Oligonucleotide OX according to claim 1, characterized in that it comprises 9 to 42 nucleotides and is capable of hybridizing under stringent conditions with an oligonucleotide OY of the sequence Y1-Y2-Y3-Y4-Y5, in which Y1 represents a nucleotide sequence of 1 to 12 nucleotides or Y1 is suppressed, Y2 represents a trinucleotide which codes for Gly, Y3 and Y4 independently represent a trinucleotide which codes for Arg or Lys and Y5 represents a nucleotide sequence of 1 to 21 nucleotides or Y5 is suppressed.

3. Oligonucleotide OX according to claim 1 or 2, characterized in that Y1 is suppressed in the oligonucleotide OY.

4. Oligonucleotide OX according to claim 1, 2 or 3, characterized in that Y5 is suppressed in the oligonucleotide OY.

5. Oligonucleotide OX according to claim 1, 2 or 3, characterized in that, in OY, Y5 represents a nucleotide sequence Y6-Y7-Y8-Y9, in which Y6 represents a trinucleotide which codes for Ser, Thr or Tyr, Y7 represents a trinucleotide which codes for any amino acid, Y8 represents a trinucleotide which codes for Glu or Asp and Y9 represents a nucleotide sequence comprising 1 to 12 nucleotides.

6. Oligonucleotide OX according to claim 5, characterized in that Y1 and Y9 are suppressed in the oligonucleotide OY.

7. Oligonucleotide OX according to claim 6, characterized in that it can hybridize with the said oligonucleotide OY in which Y2 represents a trinucleotide which codes for Gly, Y3 represents a trinucleotide which codes for Lys, Y4 represents a trinucleotide which codes for Arg and Y5 represents a sequence of 3 trinucleotides which codes for Ser-Ala-Glu.

8. Single-stranded oligonucleotide OY, characterized in that it comprises 9 to 42 nucleotides of the sequence Y1-Y2-Y3-Y4-Y5, in which Y1 represents a nucleotide sequence of 1 to 12 nucleotides or Y1 is suppressed, Y2 represents a trinucleotide which codes for Gly, Y3 and Y4 independently represent a trinucleotide which codes for Arg or Lys and Y5 represents a nucleotide sequence of 1 to 21 nucleotides or Y5 is suppressed.

9. Oligonucleotide OY according to claim 8, characterized in that Y1 is suppressed.

10. Oligonucleotide OY according to claim 8 or 9, characterized in that Y5 is suppressed.

11. Oligonucleotide OY according to claim 8 or 9, characterized in that Y5 represents a nucleotide sequence Y6-Y7-Y8-Y9, in which Y6 represents a trinucleotide which codes for Ser, Thr or Tyr, Y7 represents a trinucleotide which codes for any amino acid, Y8 represents a trinucleotide which codes for Glu or Asp and Y9 represents a nucleotide sequence comprising 1 to 12 nucleotides.

12. Oligonucleotide OY according to claim 11, characterized in that Y1 and Y9 are suppressed.

13. Oligonucleotide OY according to claim 12, characterized in that Y2 represents a trinucleotide which codes for Gly, Y3 represents a trinucleotide which codes for Lys, Y4 represents a trinucleotide which codes for Arg and Y5 represents a sequence of 3 trinucleotides which codes for Ser-Ala-Glu.

14. Single-stranded oligonucleotide OZ, characterized in that it comprises 15 to 39 nucleotides and is capable of hybridizing under mild or stringent conditions with a consensus signal sequence characteristic of amidated polypeptide hormones, the said sequence having as the formula Z1-Z2-Z3-Z4-Z5-Z6-Z7, in which Z1 represents a nucleotide sequence of 1 to 12 nucleotides or Z 1 is suppressed, Z2 and Z3 represent two trinucleotides which code for Leu, Z4 and Z5 represent two trinucleotides which code for any two amino acids, Z6 represents a trinucleotide which codes for Leu and Z7 represents a nucleotide sequence of 1 to 12 nucleotides or Z7 is suppressed.

15. Group of oligonucleotides OX according to any one of claims 1 to 7 or of oligonucleotides OZ according to claim 14, characterized in that it constitutes a combinatorial library.

16. Method for identification of the precursor of a peptide having an amidated C-terminal end, characterized by the following successive stages:
obtaining of a DNA bank;
hybridization of one or more oligonucleotides according to any one of claims 1 to 7 with the said DNA bank;
identification of the DNA sequence or sequences of the said bank which hybridizes with an oligonucleotide according to any one of claims 1 to 7;
identification in this sequence or sequences of one or more precursors of peptides with a possible amidated C-terminal end.

17. Method according to claim 16, characterized in that the hybridization stage uses a combinatorial library according to claim 15.

18. Method for identification of the precursor of a peptide having an amidated C-terminal end, characterized by the following successive stages:
obtaining of a DNA bank;
use of the PCR technique to amplify the fragment of interest with the aid of a group of oligonucleotides according to any one of claims 1 to 7 and another group of oligonucleotides according to claim 14;
identification of the DNA sequence or sequences of the said bank which hybridizes with the oligonucleotide according to any one of claims 1 to 7;
identification in this sequence or sequences of one or more precursors of peptides with a possible amidated C-terminal end.

19. Method according to claim 18, characterized in that the amplification stage uses a combinatorial library according to claim 15.

20. Method for identification of the precursor of a peptide having an amidated C-terminal end, characterized by the following successive stages:
obtaining of a DNA bank;
use of the PCR technique to amplify the fragment of interest with the aid of a group of oligonucleotides according to any one of claims 1 to 7;
identification of the DNA sequence or sequences of the said bank which hybridizes with the oligonucleotide according to any one of claims 1 to 7;
identification in this sequence or sequences of one or more precursors of peptides with a possible amidated C-terminal end.

21. Method according to claim 20, characterized in that the amplification stage uses a combinatorial library according to claim 15.

22. Method for identification of the precursor of a polypeptide having an amidated C-terminal end, characterized by the following stages:
obtaining of a DNA bank;
use of the PCR technique to amplify the fragment of interest with the aid of an oligonucleotide according to any one of claims 1 to 7 and another single-stranded oligonucleotide capable of hybridizing under mild or stringent conditions with a universal consensus sequence contained in the sequence of the plasmid vector in which the cDNA of the said DNA bank are cloned, such as the primers T3, T7, KS, SK, M13, Reverse;
identification of the DNA sequence of the said bank which hybridizes with an oligonucleotide according to any one of claims 1 to 7;
identification in this sequence of one or more precursors of peptides with a possible amidated C-terminal end.

23. Method according to claim 22, characterized in that the amplification stage uses a combinatorial library according to claim 15.

24. Method according to any one of claims 16 to 23, characterized in that the DNA bank is cDNA bank.

25. Method according to any one of claims 16 to 24, characterized in that the single-stranded oligonucleotide can be detected with the aid of a marking agent, such as ³²P or digoxigenin.
